(19) United States
(12) Patent Application Publication
(10) Pub. No.: US 2025/0336529 A1
(43) Pub. Date: Oct. 30, 2025

(54) ARTIFICIAL INTELLIGENCE BASED SYSTEM AND METHODS FOR ENDOMETRIOSIS DIAGNOSIS

(71) Applicant: HER HEALTH AI LLC, Orlando, FL (US)

(72) Inventors: Bruno DUARTE GOMES, Belém (BR); Arthur DE CASTRO NASCIMENTO, São Paulo (BR); Joao Marcos GUIMARAES, Guarulhos (BR); Joao Bosco Paulo CAMEIRO, Sao Paulo (BR); Cinthia BARBASIAN, São Paulo (BR); Henrique ABRAO, Sao Paulo (BR); Ronan Morias ROCHA, São Paulo (BR); Evandro BARROS, Barrie (CA); Arthur Pedro NETO, Belem (BR); Anderson VIANNA, Castanhal-Pará (BR); Clarrisse RUFINO, Rio de Janeiro (BR); Siddharth JAIN, Amherst, MA (US); Joelle MCCULLEN, Harvard, IL (US); Akifa KHATTAK, Orlando, FL (US); Marina DE PAULA ANDRES AMARAL, SAO PAULO (BR); Mauricio ABRAO, SAO PAULO (BR); Ramanathan Anandarama KRISHNAN, Chennai (IN)

(21) Appl. No.: 19/191,901

(22) Filed: Apr. 28, 2025

Related U.S. Application Data
(60) Provisional application No. 63/639,276, filed on Apr. 26, 2024.

(51) Int. Cl. G16H 50/20; G06N 20/00; G06T 7/00; G16H 10/60; G16H 30/00; G16H 50/30

(52) U.S. Cl. CPC G16H 50/20; G06N 20/00; G06T 7/0012; G16H 10/60; G16H 30/00; G16H 50/30; G06T 2207/10088; G06T 2207/20081; G06T 2207/30096

(57) ABSTRACT

A computer-implemented method of endometriosis diagnosis includes applying, by the computer system, one or more machine learning models configured to analyze training data comprising pelvic medical images and clinical data that include both patients diagnosed with endometriosis, and patients not diagnosed with endometriosis. The machine learning algorithm is configured to determine a probability of a hypothetical patient belonging to a group with endometriosis based on a plurality of rules developed from patterns learned from the training data. The computer-implemented method also includes acquiring clinical data for a particular patient, acquiring pelvic medical imaging for the particular patient, and determining a probability of an endometriosis diagnosis for the particular patient using the plurality of rules from the machine learning algorithm and the clinical data and pelvic medical imaging for the particular patient.

### CROSS-REFERENCE TO RELATED APPLICATION

This application claims the benefit of U.S. Provisional Patent Application Ser. No. 63/639,276, filed Apr. 26, 2024, the contents of which are incorporated by reference herein.

### FIELD

The present invention relates to the field of medical diagnosis, and, more particularly, to artificial intelligence based system and methods for endometriosis diagnosis.

### BACKGROUND

Endometriosis is a condition impacting 6.5 million American women (˜11% of total reproductive age), and 200 million women globally. It ranks among the top 10 most painful diseases, and leads to infertility, pelvic floor dysfunction, fatigue, hormone imbalance, early menopause, fibroids, adhesions, adenomyosis, ovarian cysts, as well as other physical symptoms. Despite a prevalence comparable to diabetes, endometriosis is marked by disparities in research funding—less than 2 percent of total R&D expenditure goes to endometriosis. This disease is prevalent in women aged 15 to 44, with a higher incidence in those in their 30s and 40s. Endometriosis does not fall under ADA work accommodations as a disability, thus contributing to the $1.8 billion lost productivity and earnings the US economy from lack of accommodations.

Adenomyosis, closely related to endometriosis and also impacting ovarian health, is typically found in women in their 40s and 50s. Adenomyosis is when uterine lining tissue grows into the muscular wall, causing heavy bleeding and pelvic pain, while endometriosis involves similar tissue growing outside the uterus. Adenomyosis and endometriosis can coexist and may exacerbate symptoms of pelvic pain and heavy menstrual bleeding when present together. Despite the widespread prevalence of these conditions, the diagnostic process typically spans a staggering seven to eleven years through the gold standard-surgery. This is because of difficulties in detecting endometriosis with blood tests and medical imaging. The delayed diagnosis leads to prolonged suffering (50% of women experience suicidal thoughts), decreased quality of life, and unnecessary surgical procedures, in addition to delaying treatment and management of symptoms. There is a clear unmet need for tools that surgeons can utilize to identify endometriosis earlier than the current timeline.

The current standard for diagnosing endometriosis primarily relies on invasive procedures such as laparoscopy with biopsy, which is considered the gold standard for diagnosis and staging. Diagnosing endometriosis via blood markers and biopsies have been known for the past thirty years but has its major limitations. CA125's lack of specificity (specificity 0.78 to 0.98, sensitivity ranges 0.23 to 0.93) due to elevation in conditions like ovarian cysts and pelvic inflammatory disease, coupled with the multifaceted nature of endometriosis, necessitates the development of more reliable, specific diagnostic tools incorporating clinical symptoms, imaging, and multiple biomarkers for accurate diagnosis. Similarly, HE4 has a sensitivity of approx. 0.50 to 0.90 and specificity approx. 0.78 to 0.95, CRP has a sensitivity 0.30 to 0.70 and specificity 0.63 to 0.79, and AMH is typically to determine ovarian reserve for IVF.

Moreover, endometriosis lesions may be present despite negative blood results. These methods also remain invasive and do not map the sites of endometriosis, which is essential for the optimal surgical outcomes. Although recent advances in imaging tests, transvaginal and pelvic ultrasound (TVUS) and pelvic magnetic resonance imaging (MRI), have shown promise in improving diagnostic accuracy, they still rely heavily on the expertise of healthcare professionals for interpretation. Despite these advancements, significant challenges persist with these methods in achieving timely and accurate diagnosis due to factors such as the absence of clinical suspicion, limited availability of specialized imaging tests, lack of consistent protocols for preparation of imaging examinations, and the complexity of clinical presentation in endometriosis. After attending the 2024 Endometriosis Summit, endometriosis surgeons primary concern was how to locate lesions with varied appearances, however, there was minimal consensus.

While some studies have explored the potential of artificial intelligence (AI) in improving diagnostic accuracy for endometriosis, its application in this field remains limited. Existing AI models focus on predictive and diagnostic models using clinical variables and symptoms. Recent studies have demonstrated advancements in endometriosis diagnosis and management. Goncalves, Siufi Neto, Andres, Siufi et al. (2021) highlighted the effectiveness of imaging tests like transvaginal ultrasound (TVUS) in diagnosing ovarian and deep endometriosis. Chattot et al. (2019) utilized AI to refine surgical eligibility criteria for deep infiltrative endometriosis patients, showcasing AI's potential in decision-making. Maicus et al. (2021) achieved high accuracy in classifying the state of the Douglas pouch using an AI deep learning model. Akter et al. (2019) evaluated genes in transcriptomics and methylated data with high accuracy. Additionally, Sivajohan et al. (2022) explored various AI applications in endometriosis diagnosis and prediction, reporting pooled sensitivities ranging from 81.7% to 96.7% and specificities between 70.7% and 91.6%. Despite these advancements, a key technical challenge remains effectively integrating disease-relevant imaging variables and other clinical data into predictive and diagnostic AI models.

### SUMMARY

A computing system for endometriosis diagnosis is disclosed. The computing system includes at least one processor, and a memory storing instructions that, when executed by the at least one processor, causes the system to execute a machine learning algorithm. The machine learning algorithm is configured to analyze training data comprising pelvic medical images and clinical data that include both patients diagnosed with endometriosis, and patients not diagnosed with endometriosis. The machine learning algorithm is configured to determine a probability of a hypothetical patient belonging to a group with endometriosis based on a plurality of rules developed from patterns learned from the training data. In addition, the system includes acquiring clinical data for a particular patient, acquiring pelvic medical imaging for the particular patient, and determining a probability of an endometriosis diagnosis for the particular patient using the plurality of rules from the machine learning algorithm and the clinical data and pelvic medical imaging for the particular patient. The clinical data may include lower back pain, bloating, dysmenorrhea, fatigue, vaginal touch, infertility, pain before period, dyspareunia, pain during period, and regular stomach pain.

The machine learning algorithm comprises one of logistic regression, Random Forest, and XG Boost, and the medical imaging comprises a magnetic resonance imaging (MRI) image or an ultrasound image, where the MRI image is cropped to capture the volume of interest. In addition, bias field correction may be applied to the MRI image, and a spatially adaptive filter may be applied to the MRI image to attenuate noise registered in the MRI image during scanning. Voxel values of the MRI image may be scaled to a controlled range to further improve quality of the image.

In addition, the computing system may be configured to analyze the clinical data for the particular patient to generate a clinical based diagnosis, and to process the medical imaging to produce an imaging based diagnosis.

In another particular aspect, a computer-implemented method of endometriosis diagnosis is disclosed. The computer-implemented method includes applying, by the computer system, one or more machine learning models to analyze training data comprising pelvic medical images and clinical data that includes both patients diagnosed with endometriosis, and patients not diagnosed with endometriosis. The machine learning algorithm is configured to determine a probability of a hypothetical patient belonging to a group with endometriosis based on a plurality of rules developed from patterns learned from the training data. The computer-implemented method also includes acquiring clinical data for a particular patient, acquiring pelvic medical imaging for the particular patient, and determining a probability of an endometriosis diagnosis for the particular patient using the plurality of rules from the machine learning algorithm and the clinical data and pelvic medical imaging for the particular patient.

In yet another aspect, a non-transitory computer-readable storage medium including instructions that, when executed by at least one processor of a computing system, cause the computing system to perform a method comprising applying one or more machine learning models to analyze training pelvic medical images and clinical data that include both patients diagnosed with endometriosis, and patients not diagnosed with endometriosis. The instructions also include determining a probability of a hypothetical patient belonging to a group with endometriosis based on a plurality of rules developed from patterns learned from the training data.

One objective of the system is to present a novel approach to gynecological diagnostics. The novel system comprises a comprehensive endometriosis diagnostics tool utilizing AI algorithms, Software as a Medical Device (SaMD), to address this unmet need in the market.

Another objective of the system is to target demographic for the AI-driven diagnostic tool for endometriosis and its associated disease, adenomyosis, primarily includes women between the ages of 15 and 50. The system significantly improves patient outcomes by facilitating early diagnosis and aiding surgeons in achieving higher specificity and sensitivity rates. This, in turn, aims to reduce the necessity for repeat surgeries due to residual disease.

Another objective of the system is to provide a system that responds to a clear and pressing health problem: the delayed diagnosis of endometriosis, which often leads to prolonged suffering, decreased quality of life, and unnecessary surgeries. By providing a non-invasive and cost-effective diagnostic tool covered by insurance, this empowers healthcare providers to identify endometriosis earlier, leading to timely intervention and improved patient outcomes.

Another objective of the system is to catalyze significant advancements in the prevention and management of gynecological diseases, ultimately leading to better health outcomes for women globally. The system offers preoperative support, providing diagnoses and imaging endometriosis mapping reports to guide surgery and improve outcomes within existing laparoscopic workflows, integrating without adding to the surgeon's time or requiring additional training.

### BRIEF DESCRIPTION OF THE DRAWINGS

The aspects and the attendant advantages of the embodiments described herein will become more readily apparent by reference to the following detailed description when taken in conjunction with the accompanying drawings wherein:

FIG. 1 is a block diagram of an artificial intelligence based system for endometriosis diagnosis in accordance with particular aspects of the invention disclosed herein;

FIG. 2 is a schematic of an application server of the invention;

FIG. 3 is a block diagram of the application architecture of the system;

FIG. 4 is a chart of model training results according to the ROC curve metric;

FIG. 5 is a chart of the model training results according to the accuracy metric;

FIG. 6 is a chart of the model results on test data according to some metrics;

FIGS. 7-28 are graphical results to illustrate the prediction accuracy and sensitivity of the machine learning algorithms;

FIG. 29 is the code for the XG Boost machine learning algorithm;

FIG. 30 is the code for the logistic regression machine learning algorithm;

FIG. 31 is a chart of feature importance for logistic regression;

FIG. 32 is the code for the Random Forest machine learning algorithm;

FIG. 33 is a chart of the feature importance for Random Forest;

FIG. 34 is an image illustrating an example of a cropped volume of the pelvis;

FIG. 35 is an image illustrating a multimodal (CT and MRI) image matching through co-registration;

FIG. 36 is images illustrating a comparison between not corrected images, and corrected images of the pelvis;

FIG. 37 is a schematic of an architecture overview of nnU-Net;

FIG. 38 is a block diagram of a graphical user interface (“GUI”) of the system; and

FIG. 39 depicts an example computing system that can be configured to carry out the endometriosis diagnosis and machine learning model.

### DETAILED DESCRIPTION

The present invention will now be described more fully hereinafter with reference to the accompanying drawings, in which preferred embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art.

As will be appreciated by one of skill in the art upon reading the following disclosure, various aspects described herein may be embodied as a device, a method or a computer program product (e.g., a non-transitory computer-readable medium having computer executable instruction for performing the noted operations or steps). Accordingly, those aspects may take the form of an entirely hardware embodiment, an entirely software embodiment or an embodiment combining software and hardware aspects.

Furthermore, such aspects may take the form of a computer program product stored by one or more computer-readable storage media having computer-readable program code, or instructions, embodied in or on the storage media. Any suitable computer readable storage media may be utilized, including hard disks, CD-ROMs, optical storage devices, magnetic storage devices, and/or any combination thereof.

An artificial intelligence-based system for endometriosis diagnosis is disclosed. An ideal AI-based approach as claimed herein integrates diverse imaging variables, offering personalized treatment recommendations and improving patient outcomes.

One new aspect about the claimed approach of the system and method is the utilization of advanced trainable AI models, a capability that has significantly evolved in recent years. AI models were once limited in their capacity, but today's technology offers unprecedented potential for complex data analysis and pattern recognition. While existing methods rely on invasive procedures or limited imaging techniques, the claimed approach integrates a multidimensional array of data sources, including physical symptoms, medical history, medication, biopsies, blood markers, genetics, as well as imaging. By harnessing AI to analyze this comprehensive dataset, a level of diagnostic accuracy and precision is achieved that was previously unattainable.

In the clinical and laboratory phase, the claimed AI system is configured to implement supervised learning techniques to analyze diverse datasets encompassing patient demographics, medical histories, physical examination findings, laboratory test results, and histopathological reports. These datasets are preprocessed to extract relevant features and normalize data, ensuring compatibility for training.

Clinical data and pelvic MRI are used to differentiate patients. The data is used to train the algorithms that make up the machine learning model using artificial neural networks. Artificial neural networks are computational models inspired by the structure and function of biological neural networks, capable of learning complex patterns and relationships from data to perform tasks such as classification, regression, and pattern recognition.

The system uses two classes: patients diagnosed with endometriosis, and patients not diagnosed with endometriosis. The algorithm is configured to perform a binary classification task. In this case, the algorithms will be within the paradigm of supervised learning. The result of the application of the algorithms includes numbers that indicate the probability of the patient having endometriosis. For the choice of algorithms to be applied, it is fundamental to know about the complexity of the data. The data from the clinical analysis (screening), which are cyclic pelvic pain, infertility, age, are tabulated (or structured) data. The data from MRI images are unstructured. In the initial training sessions of the algorithm, 70% of the data is used for testing and 30% for validation. In the entire study, the free Anaconda Python package (Continuum analytics) was used for the backend—programming and training of the model. For the front end, which corresponds to a graphical user interface discussed in more detail below, a web application style may be created using the Tailwindcss toolkit.

For a systematic understanding of the algorithm's performance, several metrics are implemented in conjunction with commonly metrics used in the literature employing ML in the health sector: (1) accuracy corresponds to the percentage of correct classifications of the total number of classifications made. A total accuracy and an accuracy per class is determined. The total is the average of the accuracies per class. An accuracy of 92% or higher is considered acceptable, based on the best results reported in the literature; (2) sensitivity corresponds to the model's ability to indicate whether a patient has a disease. It is the ratio TP/(TP+FN). Where TP=true positive; FN=false negative. Sensitivity greater than 90% will be accepted as good; (3) specificity corresponds to the ability of the model to exclude individuals who do not have a disease or disorder. It is calculated by the ratio TN/(TN+FP). Specificity greater than 90% will be accepted as good; (4) areas under ROC (Receiver Operating Characteristic) curves, a very robust graphical method is used to evaluate the performance of the model in making the diagnosis. It corresponds to plotting sensitivity versus 1-specificity.

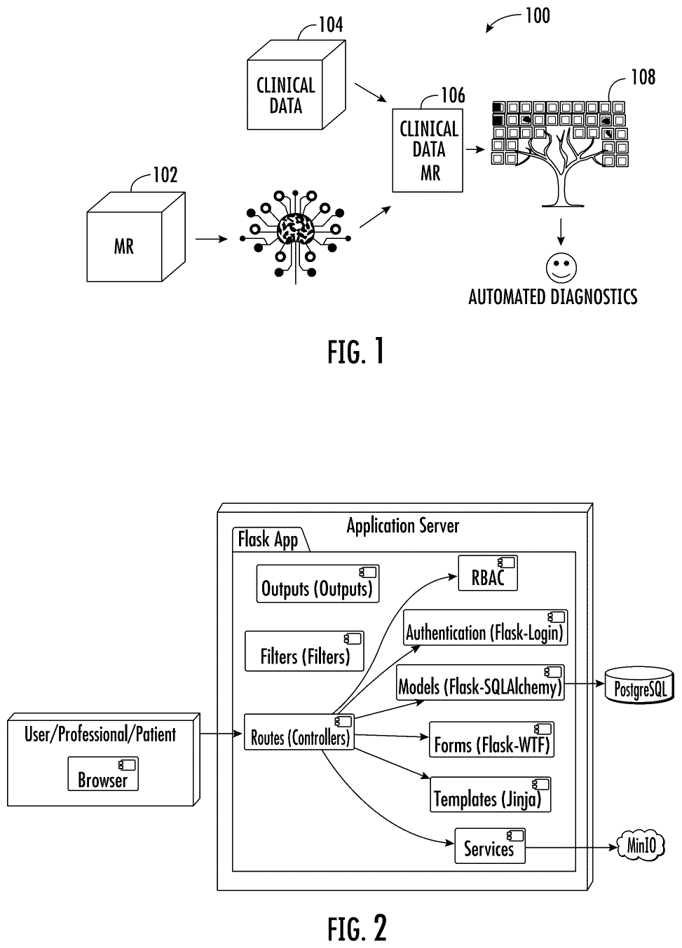

Referring now to FIG. 1, an approach for development of a machine learning model to aid in accurate, efficient diagnosis of endometriosis of the present invention is depicted and generally designated 100. Unstructured data (MRI) 102 is processed by a model based on artificial neural networks with a proprietary architecture based on U-net. Clinical data 104, corresponding to cyclic pelvic pain, infertility, age, ultrasound report, comprises tabulated data. Neural network output 106 will be probability of the patient belonging to the group with endometriosis. Total data, clinical plus the neural network's conclusion about the MRI, now grouped as structured data, will be the input for a decision tree 108 in the XGBoost learning model (or other model). The output of the tree is the probability of the patient having endometriosis.

The application architecture explains how the components of the software system are organized and how they interact with one another as depicted in FIG. 2. It is the skeleton” of the application, defining its structure and behavior. The Folder Structure includes:
app/models: contains the classes that define the data models;
app/forms: contains the classes that define the web forms;
app/filters: contains custom filters for templates;
app/routes: contains the applica8on routes and controllers;
app/services: contains the business logic of the application;
app/static: contains static files such as CSS, JavaScript, and images; and
app/templates: contains the HTML templates.

Technologies Used include:
Python: programming language;
Flask: Python web framework;
Flask-SQLAlchemy: Integration with the SQLAlchemy database;
Flask-Login: manages user authentication;
Flask-WTF: web form creation;
WTForms: defines form fields and validation;
MinIO: object storage server compatible with Amazon S3;
Postgres: relational database;
psycopg2: driver for PostgreSQL connection;
python-dotenv: loads environment variables from the .env file;
babel: internationalization and localization;
python-dateu8l: date manipulation;
Werkzeug: WSGI server for development;
SQLAlchemy: Python ORM (Object-Relational Mapper);
pydicom: DICOM file manipulation

The application follows a layered MVC architectural pattern, with the following layers:
View: responsible for the user interface (UI) and interaction with the user;
Controller: contains the application's business logic, validation rules, and data processing; and
Model: responsible for accessing and managing the application data.

The data flows between the layers of the application as follows:
1. The user interacts with the presentation layer by inputting or requesting data;
2. The presentation layer sends the data to the business layer;
3. The business layer processes the data, applies validation rules, and sends the data to the data layer;
4. The data layer stores or retrieves the data from the database;
5. The data layer returns the data to the business layer;
6. The business layer formats the data and sends it to the presentation layer; and
7. The presentation layer displays the data to the user.

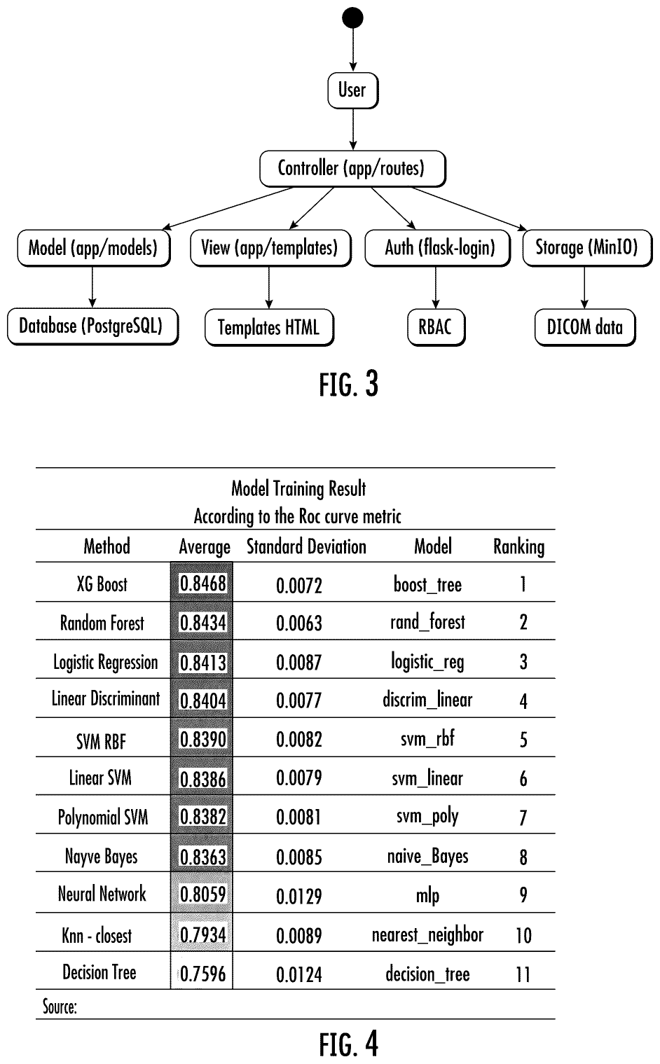

A block diagram of the Application Architecture is depicted in FIG. 3. For example, the User interacts with the application through a web browser. The Controller (app/routes) receives user requests, processes them, and interacts with the models and views and defines the application routes and manages the data flow. The Model (app/models) represents the application's data structure and interacts with the database. It also defines entities and their relationships, using Flask-SQLAlchemy for object-relational mapping (ORM). The View (app/templates) is responsible for displaying information to the user and uses HTML templates to dynamically generate web pages. The Authentication (flask-login) manages user authentication and authorization, controlling access to the application's resources. RBAC Management implements role-based access control, and defining permissions for different user groups. The Database (PostgreSQL) stores application data, such as user information, medical data, etc. The Storage (MinIO) stores DICOM files, which are medical images.

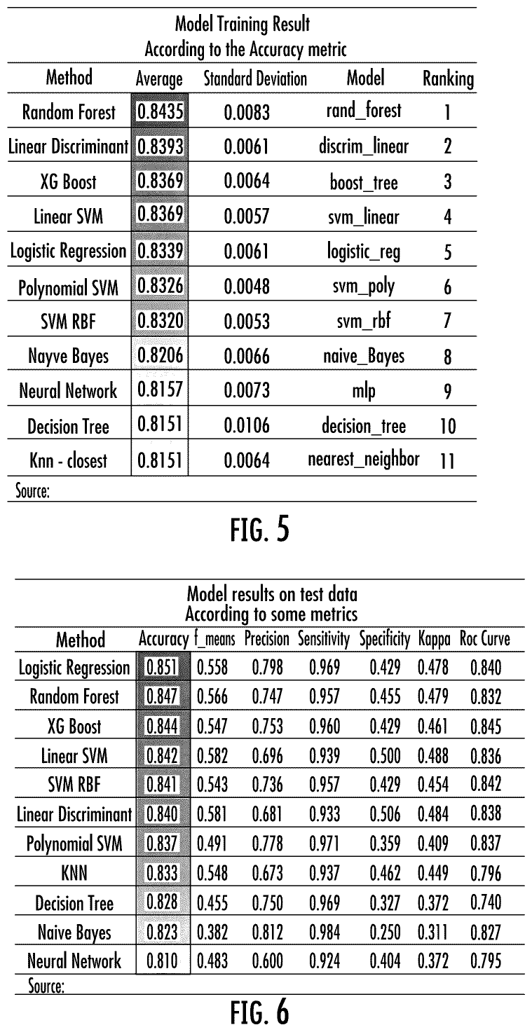

Various machine learning algorithms were assessed to determine accuracy as a predictive model for the diagnosis of endometriosis. This includes Logistic Regression, Random Forest, XG Boost, linear SVM, SVM RBF, Linear Discriminant, Polynomial SVM, KNN, Decision Tree, Naïve Bayes, and Neural Network. A summary of the model training results according to the ROC curve metric are shown in FIG. 4. A summary of the model training results according to the Accuracy metric are shown in FIG. 5.

Subsequently, the following measure were calculated on the test set: Accuracy, ROC Curve, F-measure, Precision, Sensitivity, Specificity, and Kappa. The results of applying the data to the training set, the metrics for analyzing the best method, which will be applied to the data below. Accordingly, a summary of the model results on test data according to the various metrics are shown in FIG. 6.

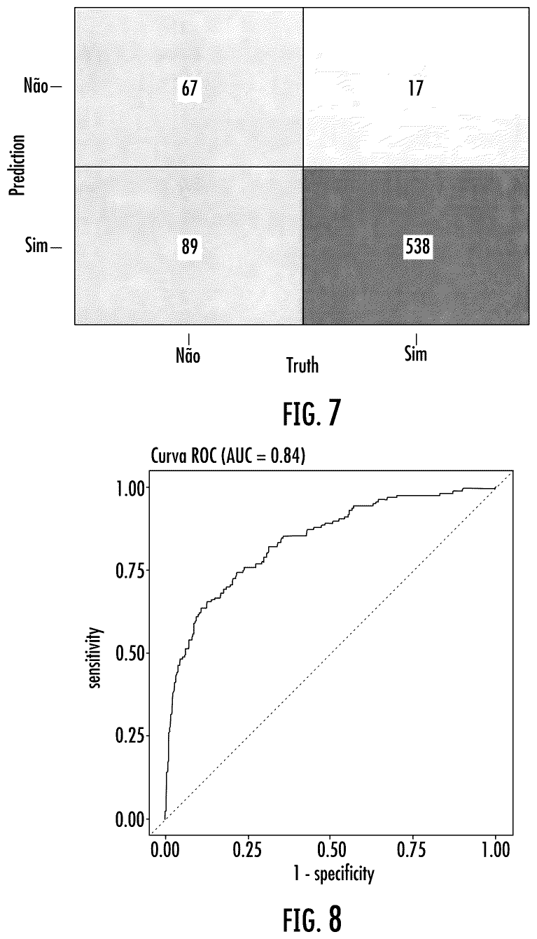

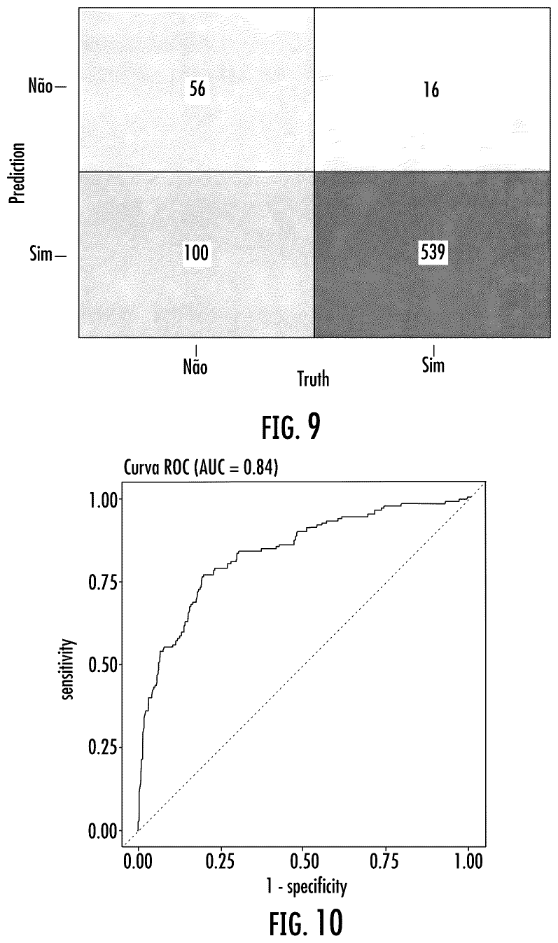

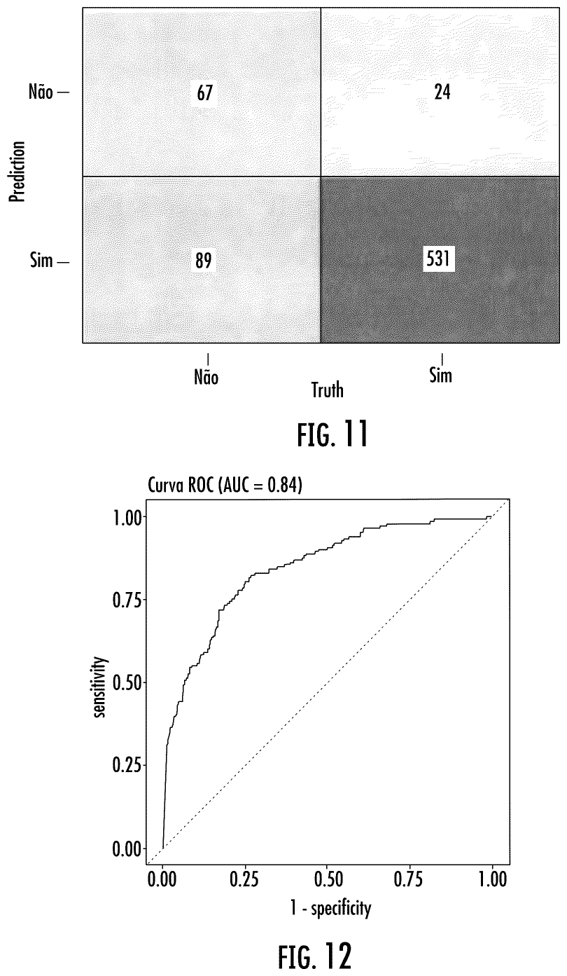

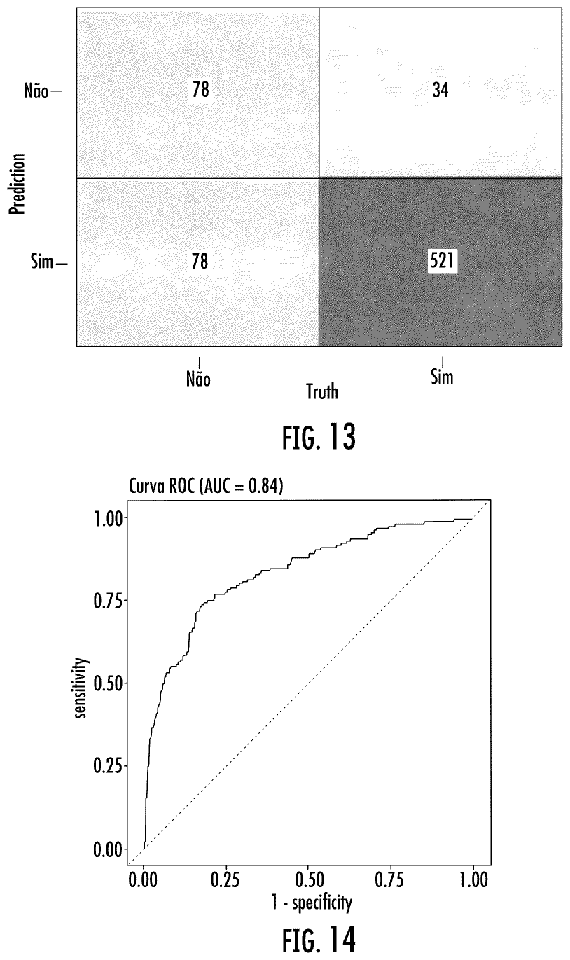

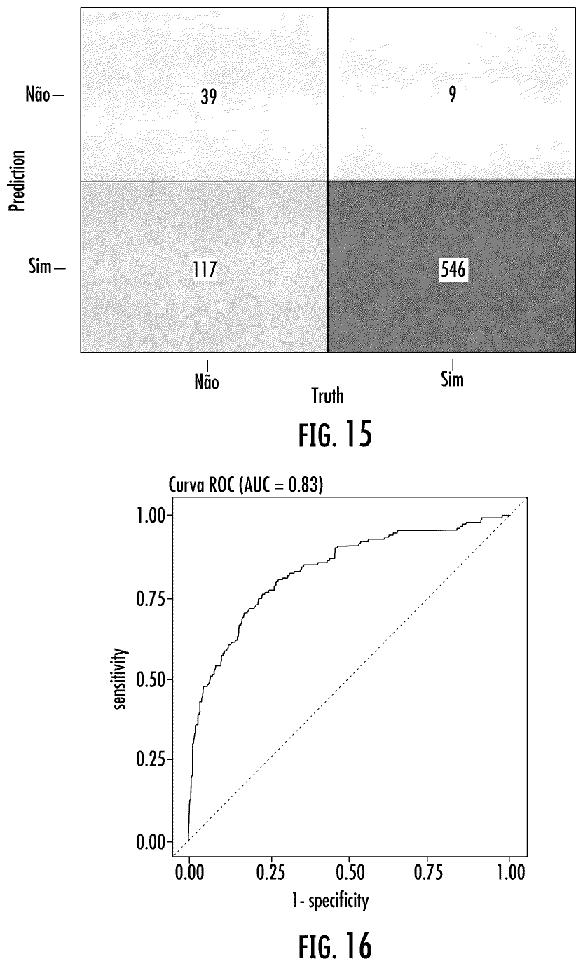

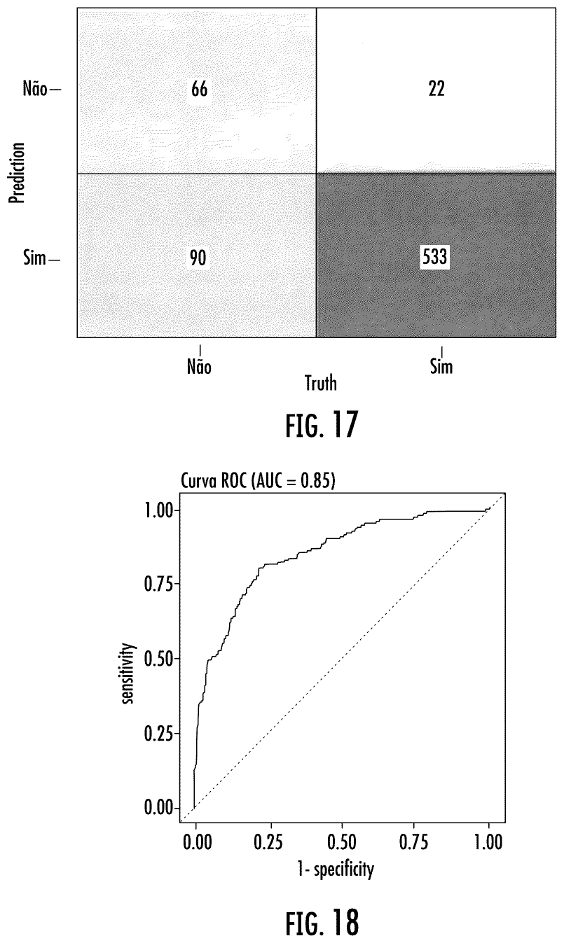

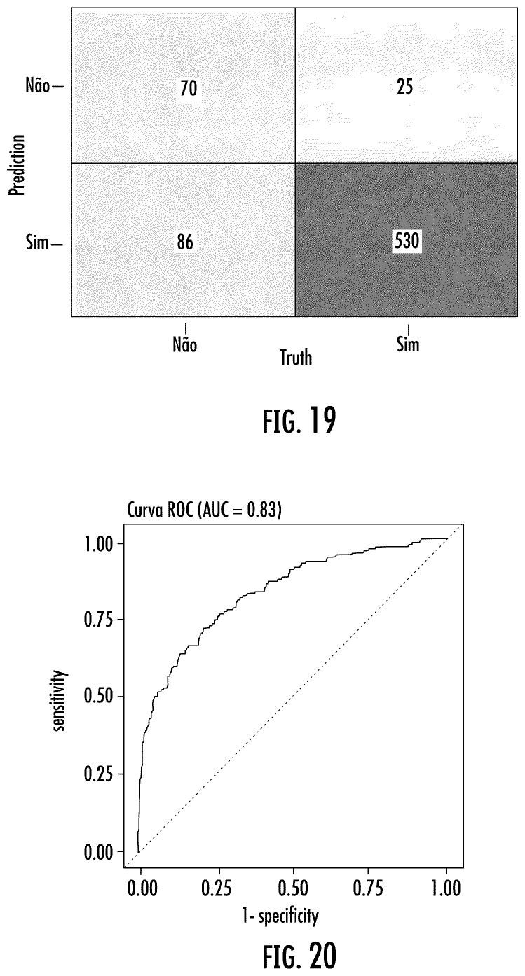

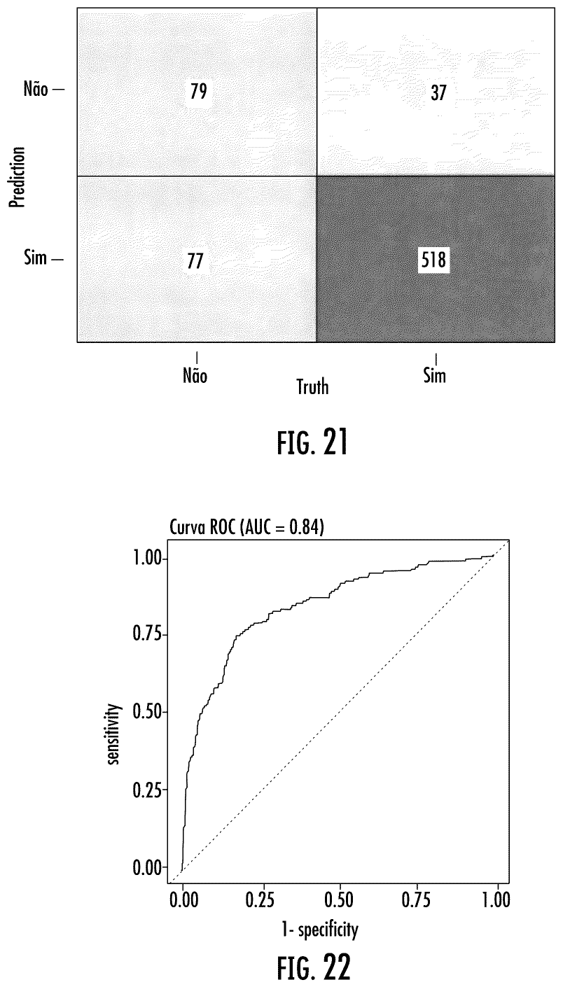

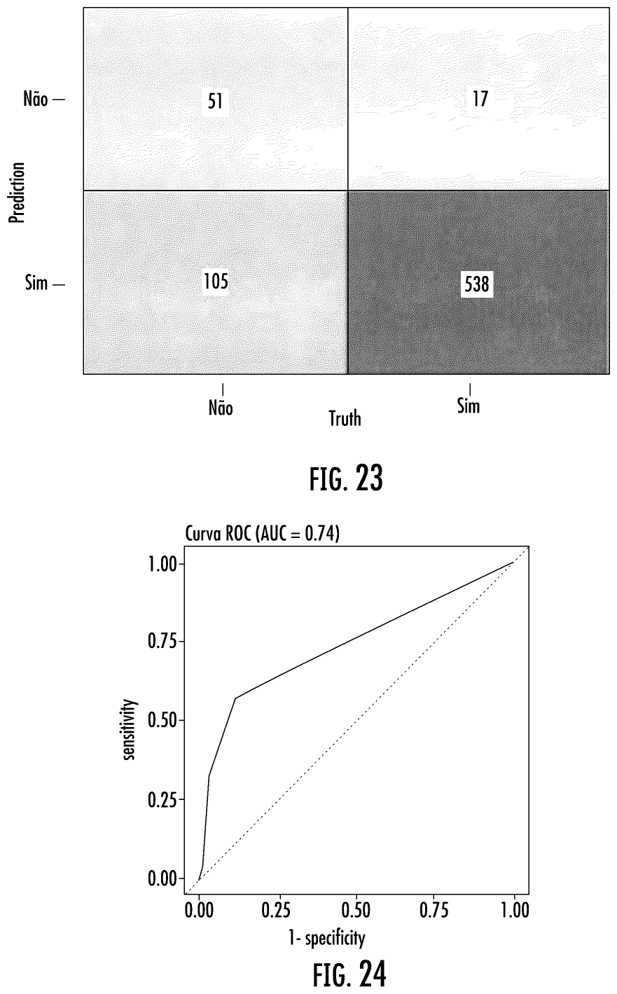

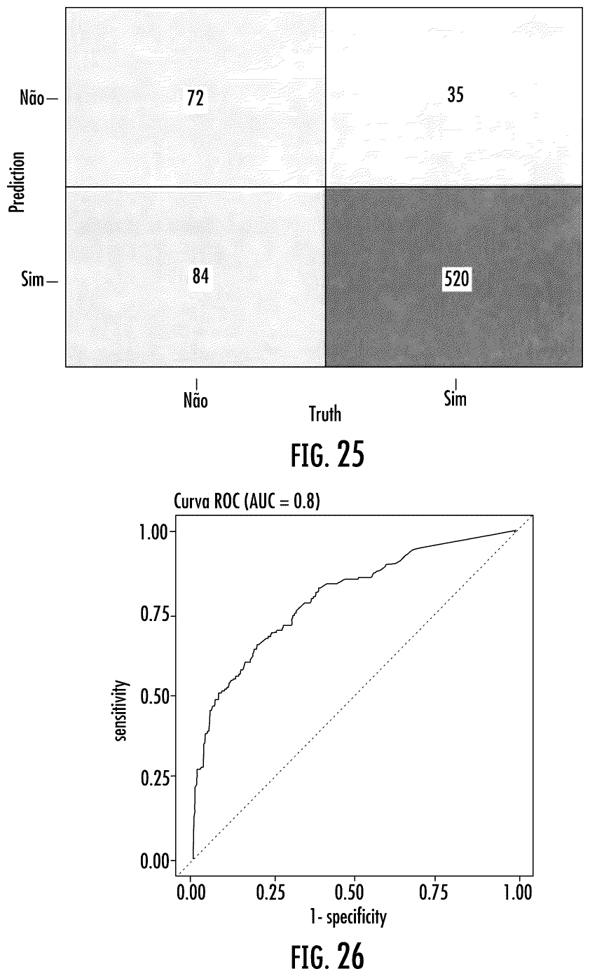

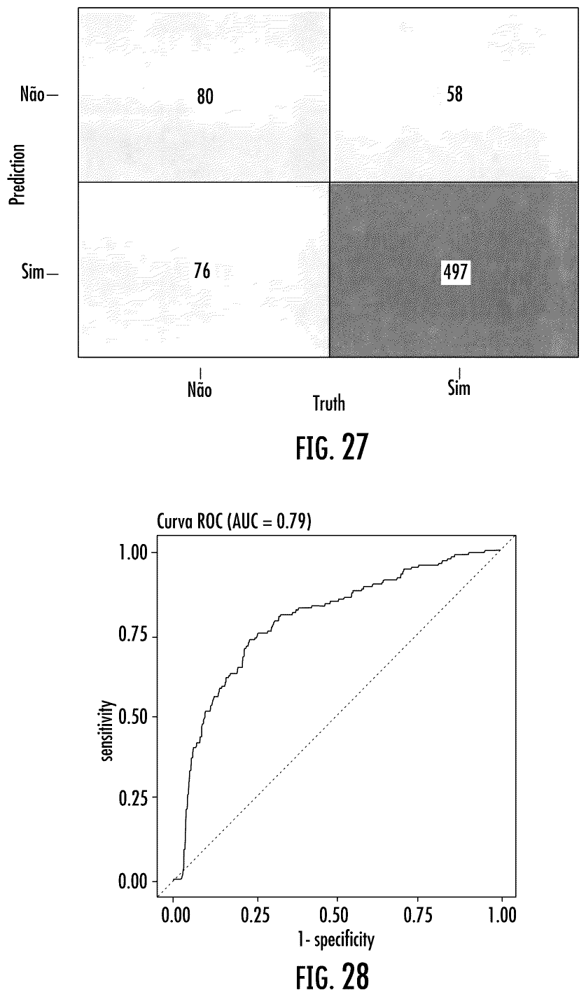

In addition, graphical results are shown in FIGS. 7-28 to illustrate the prediction accuracy and sensitivity of Logistic Regression (FIGS. 7-8), Random Forest (FIGS. 9-10), XG Boost (FIGS. 11-12), Linear SVM (FIGS. 13-14), SVM RBF (FIGS. 15-16), Linear Discriminant (FIGS. 17-18), Polynomial SVM (FIGS. 19-20), KNN (FIGS. 21-22), Decision Tree (FIGS. 23-24), Naive Bayes (FIGS. 25-26), and Neural Network (FIGS. 27-28).

Accordingly, the system comprises an AI-driven diagnostic tool for endometriosis, and holds significant commercial viability as it addresses a pressing unmet need in women's healthcare while offering tangible benefits to key stakeholders. With an estimated 6.5 million women of reproductive age in the United States alone affected by endometriosis, 200 million globally, there exists a substantial market demand for accurate and timely diagnostic solutions. The scalability of the system is beyond endometriosis, with the potential to expand into other critical areas of women's health, including various gynecological conditions, ultimately improving healthcare outcomes for women across a spectrum of health concerns.

Patients benefit from quicker diagnoses, personalized treatment plans, and improved quality of life. Healthcare providers experience enhanced diagnostic accuracy, streamlined workflows, and better patient outcomes. Payers see reduced healthcare costs through decreased reliance on invasive procedures and unnecessary treatments, as well as promote equitable access to high-quality care.

The novel system and methods described herein address the critical need for accurate endometriosis diagnosis by offering a low-cost, non-invasive alternative to diagnostic laparoscopic surgery, thus enhancing accessibility and affordability for patients. By leveraging AI technology, diagnostic accuracy is improved leading to personalized treatment plans and better patient outcomes. The approach of the system and methods includes insurance coverage, interoperability with healthcare systems, and a pilot program to validate effectiveness.

Even a small improvement in endometriosis diagnosis can significantly impact millions of women worldwide. For example, with 200 million affected, a mere 10% enhancement would positively affect 20 million lives. This highlights the urgency of continued research and innovation to improve patient outcomes. Furthermore, the claimed system and methods align with value-based care initiatives by optimizing resource allocation, improving patient outcomes, and promoting cost-effective interventions for hospital systems.

As discussed above, the present system and method uses advanced AI algorithms to integrate data from clinical symptoms, imaging, and blood markers to detect patterns associated with endometriosis. Key biological markers such as CA-125, HE4, and CRP indicate inflammation and endometrial tissue, while MRI offers structural insights.

By applying supervised machine learning, the AI model differentiates between patients with and without endometriosis, enabling diagnoses that are earlier, more reliable, simplistic, and more accurate, which will reduce delays and eliminate the need for costly and invasive diagnostic surgeries. In the clinical and laboratory phases, the AI system uses supervised learning to analyze datasets that include patient demographics, medical histories, physical exams, lab results, and histopathological reports. These datasets are pre-processed to extract key features and normalize data for training as discussed below.

For the MRI phase, both clinical data and pelvic MRI scans are used to train the machine learning algorithms. Artificial neural networks (ANNs) model the data to perform tasks like classification, regression, and pattern recognition. The system uses binary classification, distinguishing between patients with and without endometriosis. Structured clinical data, such as pelvic pain, infertility, and age, is combined with unstructured MRI data to assess the complexity of the condition. The model may be trained on 70% of the data, while 30% is used for validation. The back end may use the Anaconda Python package for programming and model training, and the front end may be built using Tailwindcss, for example.

The system utilizes automated rectosigmoid (ROI) extraction, employing template matching and k-means clustering to optimize computational efficiency. The classification pipeline integrates deep learning models such as VGG-16, VGG-19, DenseNet-121, and Xception with Recurrent Convolutional Layers (RCL) to capture spatial dependencies.

XGBoost may be employed for final classification, utilizing soft voting for robust predictions. For lesion segmentation, a two-stage approach integrates TransUNet and Vision Transformers with U-Net for precise localization. Dice loss optimizes segmentation, entropy-based active learning refines sample selection, and Monte Carlo Dropout with Grad-CAM visualizations enhances explainability and clinical confidence. In particular, the system has been demonstrated to accurately detect, diagnose, and/or monitor endometriosis in pre-clinical models or in adults and/or adolescents through its data with 98% accuracy in training non-clinical validation.

The data collection process for the system is multimodal, integrating structured clinical data and unstructured imaging data. Clinical inputs, such as patient history, symptoms, and lab results are collected during routine consultations, while pelvic MRI scans visualize endometriotic lesions. The AI model pre-processes this data by normalizing clinical inputs and applying image processing to extract regions of interest (ROIs) from the MRI scans, focusing on potential lesions.

For analysis, data is fed into an ANN, specifically a modified U-net architecture for medical image segmentation. Clinical data, including age, cyclic pelvic pain, and infertility history is processed through an XGBoost classifier for final classification. By combining imaging and clinical variables, the AI model predicts the likelihood of endometriosis, offering results as probability scores along with lesion segmentation maps, confidence intervals, and risk stratification to aid clinicians in treatment planning.

The AI model of the system is configured to provide real-time diagnostic results based on the input of clinical data, imaging, and laboratory results, allowing for faster clinical decision-making with reliance on the appropriate baseline information. For instance, once an MRI scan is completed, the system is configured to process the images, perform segmentation, and to provide a diagnostic probability score within minutes. This shortens the overall diagnostic timeline and ensures that clinicians can initiate treatment or refer patients for surgery more quickly than with traditional diagnostic methods, and improve infertility rates, for example, that are due to endometriosis.

The system has been demonstrated in several pre-clinical studies and pilots. Preliminary data shows that integrating MRI scans with clinical markers using AI significantly improves diagnostic accuracy. Internal studies utilizing deep learning models for MRI analysis achieved sensitivity rates above 90%, outperforming traditional diagnostic methods. The system AI model's lesion segmentation capability was validated using the TransUNet architecture, which employs a custom loss function combining Binary Cross-Entropy and Tversky loss to address class imbalance that is common in medical datasets with low lesion prevalence.

Early tests of the XGBoost classifier for clinical data also achieved high specificity in identifying endometriosis in women with cyclic pelvic pain and infertility. Trained on a dataset, the model reached classification accuracy rates exceeding 92% as explained below. Performance metrics, including the area under the ROC curve (AUC), showed robust results, with an AUC above 0.90, considered excellent for medical diagnostics.

The system enhances existing MRI capabilities by integrating AI to improve diagnostic accuracy and non-invasive diagnosis of endometriosis. Traditional MRI depends on expert radiologists, limiting access for many patients. By applying AI, particularly in detecting deep infiltrating endometriosis (DIE), the system makes diagnosis more accessible and precise. Recent testing of the system shows that AI-assisted imaging, using transfer learning models like VGG-16 and Xception improves the accuracy of detecting endometriotic lesions, with sensitivities of 81.7%-96.7% and specificities between 70.7%-91.6%. Preliminary pilot data shows the AI model of the system achieving 92% accuracy in lesion segmentation compared to surgical findings, and the combination of MRI with CA-125 biomarkers increases diagnostic sensitivity by 15%.

The diagnostic model of the system uses advanced AI architectures such as VGG-16, DenseNet, and Xception for feature extraction, while XGBoost combines MRI and clinical data for classification. Lesion segmentation is done using TransUNet models in a two-stage approach, providing accurate detection and refined segmentation. Active learning with entropy-based uncertainty sampling and data augmentation increases model robustness, while post-processing reduces false positives. Multi-slice analysis improves consistency across MRI slices, and Grad-CAM visualization explains the AI's decisions. Monte Carlo Dropout methods quantify uncertainty, offering clinicians confidence in the results.

The system and methods use data such as clinical symptoms, labs, imaging, and genetic markers to predict endometriosis with high accuracy. By applying machine learning, it bypasses invasive diagnostic laparoscopy, reducing the diagnostic period from 7-11 years to under one year. AI-driven analysis of MRI data enables precise, non-invasive lesion detection, improving patient safety and cutting unnecessary healthcare costs. Automated data analysis also reduces reliance on specialized healthcare workers, making diagnostics more accessible in diverse healthcare settings, including under-resourced areas. This accessibility also assists with earlier interventions for endometriosis-related infertility and cancer risks.

The system offers a scalable, cost-effective solution that can be integrated into point-of-care routine annual women's health exams, improving diagnostic accuracy and making early detection possible for the 200 million women globally. In particular, the system integrates a range of biomarkers. The primary biomarker used is CA-125, historically employed to assess advanced endometriosis. Despite its limitations in sensitivity (0.23-0.93) and specificity (0.78-0.98), particularly for early-stage disease, elevated CA-125 levels are particularly useful when combined with other data, especially in detecting deep infiltrating endometriosis (DIE).

Other key biomarkers may be integrated into the system such as Human Epididymis Protein 4 (HE4), more commonly associated with ovarian cancer diagnostics, has also shown promise for endometriosis diagnosis. With sensitivity ranging from 0.50 to 0.90 and specificity from 0.78 to 0.95, HE4 is useful for identifying more severe cases of endometriosis, adding another layer of precision to the diagnostic process. Another biomarker is C-Reactive Protein (CRP), a general marker of systemic inflammation, is often elevated in endometriosis patients. While CRP's sensitivity (0.30-0.70) and specificity (0.63-0.79) are lower compared to CA-125 and HE4, it remains valuable in assessing the patient's overall inflammatory state, which can signal disease progression. Though non-specific, CRP offers clinically actionable information regarding the systemic impact of endometriosis. Anti-Müllerian Hormone (AJH) is another biomarker although not directly indicative of endometriosis but is important for assessing ovarian reserve, particularly in patients whose ovarian function may be compromised by endometriosis. Lower AMH levels can indicate diminished ovarian function, especially relevant for those seeking fertility treatments.

In addition to these biomarkers, the system integrates imaging data from transvaginal ultrasound (TVUS) and pelvic MRI. While TVUS and MRI are standard tools for detecting ovarian endometriomas and DIE, their diagnostic accuracy depends heavily on the expertise of the operator. By incorporating advanced AI algorithms to analyze this imaging data, the system enhances detection and mapping of endometriosis lesions, overcoming variability in lesion appearance and anatomical challenges.

The system is designed to handle the complexity of diagnosing various subtypes of endometriosis, including superficial lesions, deeply infiltrating endometriosis (DIE), and ovarian endometriomas. Its AI-driven image analysis, using advanced segmentation models like TransUNet, is highly effective at identifying deeply infiltrating lesions, which are often missed by traditional imaging techniques. This makes it suitable for diagnosing DIE, which is typically more challenging due to its location and the complexity of lesion appearances.

While the AI model is trained to recognize patterns that correlate with superficial lesions, the reliance on imaging alone may reduce diagnostic sensitivity for this particular subtype. In contrast, the model has demonstrated robust performance in detecting endometriomas and mapping their extent.

The system is configured for seamless integration into existing healthcare workflows in hospitals and practices that use Epic, AthenaHealth, E-ClinicalWorks, and Oracle electronic medical record (EMR) systems, making it practical for widespread adoption. The platform can be integrated with existing hospital information systems, electronic health records, and radiology information systems, allowing for smooth data sharing and interoperability. This integration ensures that patient data, imaging results, and diagnostic outputs are readily available for supporting clinical decision-making without the need for additional software or hardware equipment.

Furthermore, the system can be integrated into pre-surgical planning workflows. For patients requiring treatment surgery, the system's ability to map endometriosis lesions and provide detailed imaging reports allows surgeons to prepare for more precise and targeted surgical interventions. By providing detailed lesion localization, the system can improve surgical outcomes and reduce the need for repeat surgeries due to missed lesions.

The system is configured to connect with existing EMRs through InterSystems IRIS for Health™, which ensures full interoperability and compliance with healthcare data standards like FHIR, HL7 V2, IHE. InterSystems IRIS for Health is used by leading independent software vendors and healthcare organizations, the largest clinical laboratories, and the largest regional health information networks. It is an extension of the InterSystems IRIS Data Platform used by Epic to support healthcare organizations whose systems count 2.5 million concurrent users, processing roughly 1.8 billion database accesses per second across all Epic customers.

InterSystems IRIS for Health is a development platform supporting i2b2 tools, OMOP CDM, OHDSI open-source tools, and scalable OMOP repository population via FHIR downloads and data transformations, while InterSystems IRIS Cloud SQL and IntegratedML offer cloud-native, high-performance DBaaS with embedded machine learning, available on a per-vCPU-hour basis through AWS and Azure. This integration improves data exchange and clinical decision-making while adhering to HIPAA and other data privacy regulations.

Another important aspect of the system is an automated semantic segmentation pipeline for T2-MRI images of patients with endometriosis. This is accomplished by leveraging advanced image processing and deep learning techniques to accurately delineate relevant regions within the pelvic anatomy, which may aid in the characterization of the disease and the planning of treatment. Initially, pre-processing steps are applied to the raw T2 MRI data to ensure consistency and enhance image quality. For this critical phase, the ANTsPy Python library Tustison et al. (2014), which is a powerful toolkit for medical image registration and analysis, may be implemented. ANTsPy is used for tasks such as bias-field correction, spatial normalization, and intensity standardization, preparing the images for optimal input into the subsequent deep learning model.

Following preprocessing, the nnU-Net framework Isensee et al. (2021) may be utilized to train a deep learning model for semantic segmentation. nnU-Net is renowned for its ability to automatically configure itself, including model architecture and training parameters, achieving state-of-the-art performance across a wide range of medical segmentation tasks. This framework is trained on a preprocessed T2 MRI dataset to learn the complex patterns associated with endometriosis and surrounding structures.

The system was tested first using synthetic data for binary classification in order to specifically predict the presence or absence of endometriosis. Real-world patient data was simulated by generating synthetic samples based on common symptoms reported in patients with or without endometriosis. The model was trained to distinguish between two classes (Control vs. Endometriosis) based on ten synthetically generated metrics of biologically relevant symptoms.

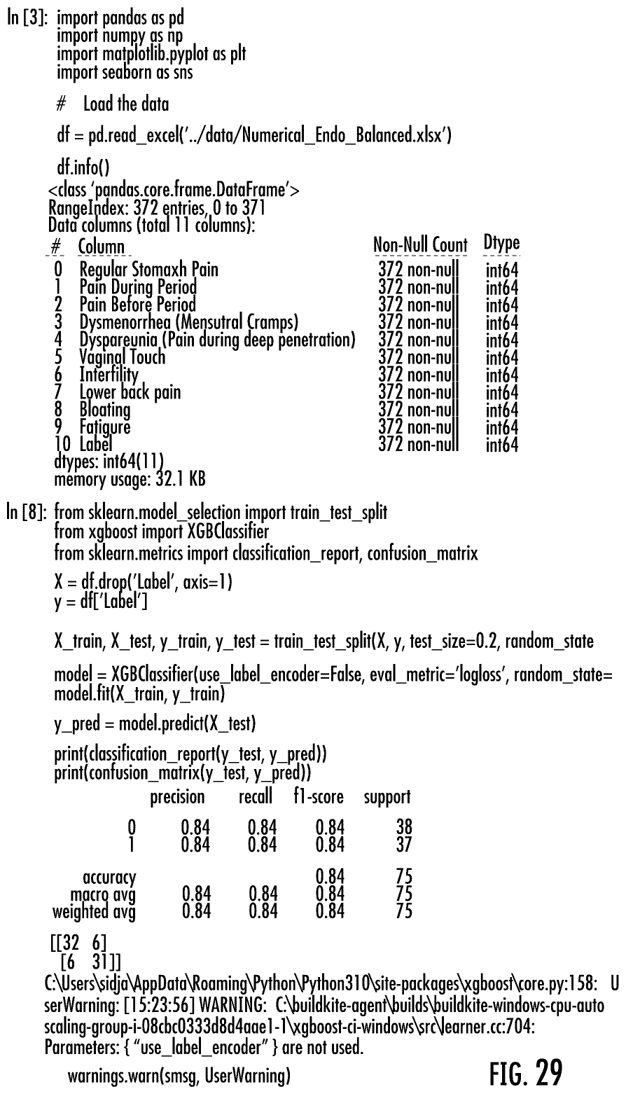

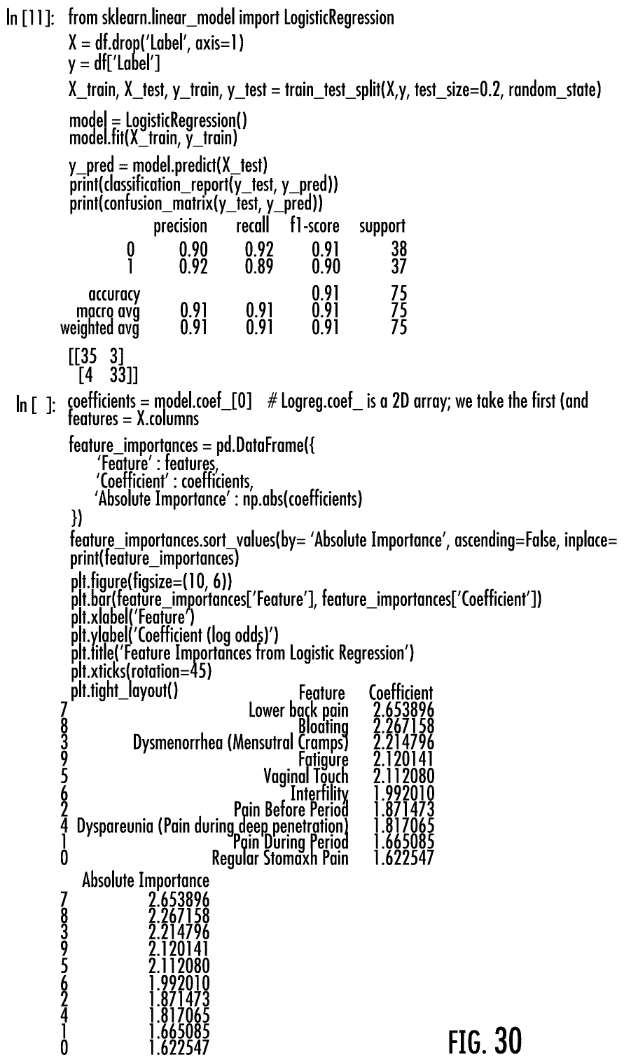

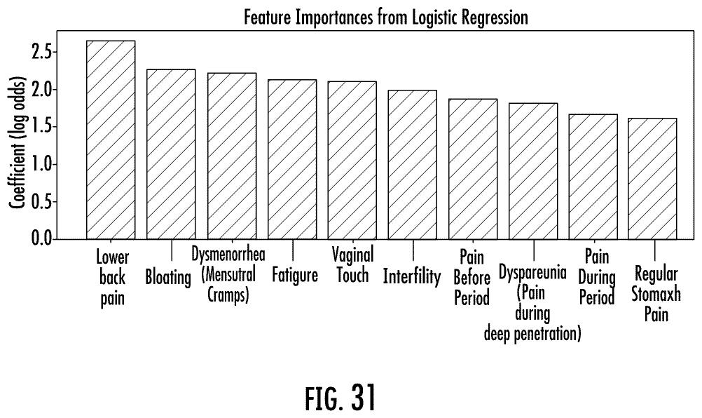

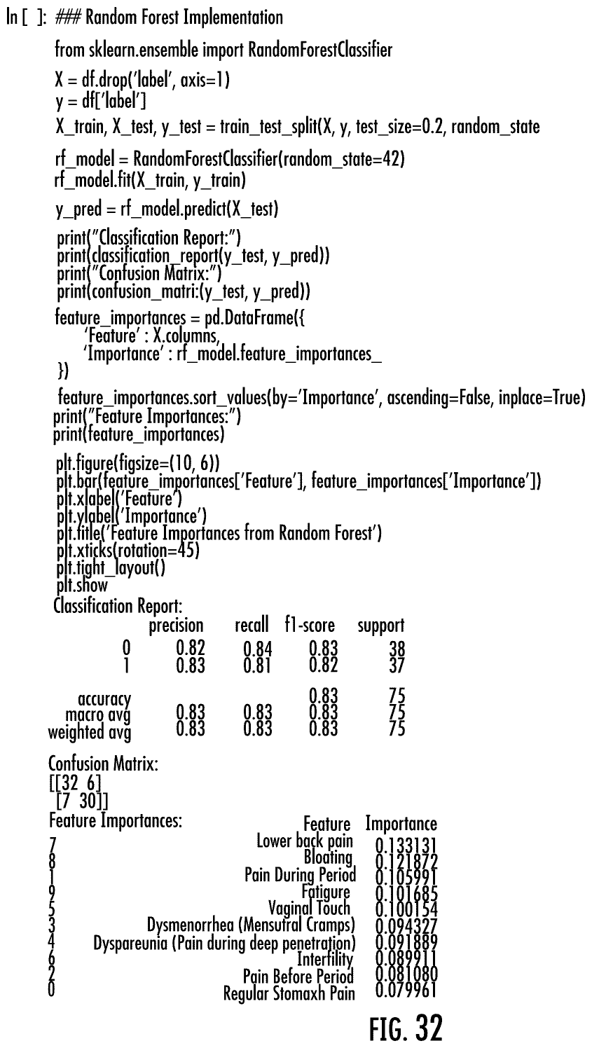

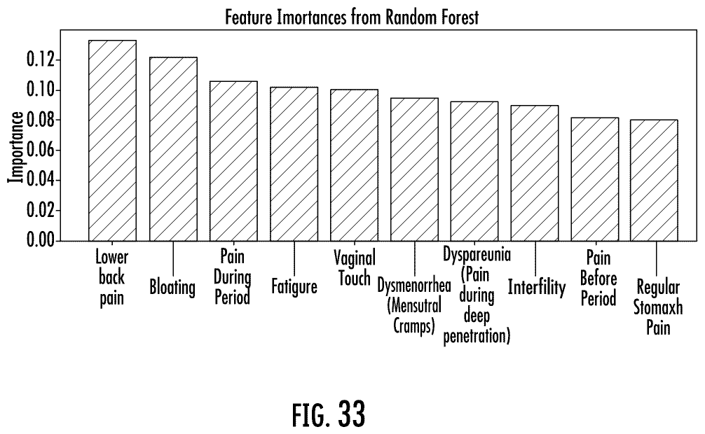

Basic models for the balanced dataset were investigated and the code for XG Boost is shown in FIG. 29, and the code for logistic regression is shown in FIG. 30. Feature importance is illustrated in FIG. 31 for logistic regression. The code of Random Forest is shown in FIG. 32 and feature importance from Random Forest is illustrated in FIG. 33. A summary of the results is as follows: Logistic Regression: 91%; XGBoost: 84%; and Random Forest: 83%.

The dataset included key symptoms often associated with endometriosis and random targets assigned as final labels (0 and 1). The symptoms were regular stomach pain, pain during periods, pain before periods, pain during deep penetration, vaginal sensitivity or touch, infertility, lower back pain, bloating, and fatigue. In addition, two separate datasets were created where one was a Balanced Dataset and included an equal number of control and endometriosis samples. The second dataset was a Causal Dataset where there was a slight imbalance between classes to simulate real-world bias.

Several machine learning models for binary classification were tested, including Random Forest, XGBoost, Support Vector Machine (SVMV), Logistic Regression, Decision Tree, Neural Networks, AdaBoost, and Naive Bayes. The goal was to assess the performance of these models on the synthetic dataset and determine which are most effective at predicting endometriosis presence.

The accuracy results from using the Balanced Dataset of 372 samples (186 Control, 186 Endometriosis), were as follows: Random Forest 88%; XG Boost 88%; Logistic Regression 92%; SVM 93%; ELM 85%; Neural Networks 93%; ADA Boost 92%; and Naive Bayes 88%.

The accuracy results from using the Causal Dataset of 300 samples (187 Control, 113 Endometriosis), were as follows: Random Forest 93%; XG Boost 93%; Logistic Regression 100%; SVM 100%; ELM 90%; Neural Networks 100%; ADA Boost 100%; and Naive Bayes 93%.

Support Vector Machines (SVMV) (Best as per Learning Curves), Neural Networks, Logistic Regression, and AdaBoost showed exceptional performance, especially on the causal dataset where all four models achieved 100% accuracy. Random Forest and XG Boost also performed consistently well across both datasets, maintaining over 90% accuracy, making them reliable backup models in practical deployments.

Additional data processing techniques were evaluated to improve the accuracy of the diagnosis using the system such as noise identification and removal from the data. The advanced pipeline employed four sophisticated clustering techniques to identify and remove noisy records. The first technique identified outliers based on local density deviations with n_neighbors=20. The next technique was OPTICS (Ordering Points to Identify the Clustering Structure) applied with min_samples=5, xi=0.05 to detect density-based noise. The third technique was K-means clustering used with n clusters=2 to identify points exceeding mean distance plus two standard deviations. The fourth technique was K-Nearest Neighbors (KNN) applied with n_neighbors=15 to identify label inconsistencies.

KNN-based noise removal proved most effective, eliminating 231 records (162 from control group, 69 from endometriosis group) that exhibited inconsistent symptom patterns. This noise removal was critical; patients in the noisy subset without endometriosis paradoxically showed higher pain levels than those with the disease, suggesting potential comorbidities like irritable bowel syndrome or adenomyosis.

The basic pipeline struggled with a significant class imbalance with the original dataset comprising 1,983 endometriosis cases vs. 563 controls (3.5:1 ratio). The basic approach has minimal adjustment, resulting in 84.7% accuracy but only 72.3% recall. The advanced pipeline implemented SMOTE for synthetic minority oversampling and cluster-based balancing to achieve 1:1 ratio. The result was 96.2% recall (p<0.001).

The basic pipeline used a broad selection of 30+ features with minimal clinical validation, while the advanced approach employed statistical feature selection comprising chi-squared tests identified statistically significant symptoms (p<0.05), clinical relevance filtering where features were ranked by both statistical significance and domain knowledge, and Random Forest Feature Importance where the model's intrinsic feature_importances_attribute quantified each symptom's contribution.

The analysis also revealed that the following pain-related symptoms had the highest predictive power: pelvic pain (acyclic), dysmenorrhea (menstrual cramps), cyclic intestinal changes, discharge, and pain level. Additionally, comorbidities including autoimmune diseases and diabetes were identified as significant predictors.

The validation strategy for the basic pipeline used a simple 70-30 train-test split, while the advanced approach implemented stratified 10-fold cross-validation to ensure robust evaluation across all metrics, external validation on independent test sets to verify generalizability, and comprehensive metric reporting with standard deviations (σ=0.018 vs. 0.032 in basic pipeline).

The advanced pipeline achieved 98.9% recall (sensitivity) with the ensemble model compared to 94.7% in the basic approach. This 4.2% improvement is clinically significant because each percentage point represents patients who would otherwise face continued diagnostic delays (7-12 years on average). The system identified 31 asymptomatic endometriosis cases missed by standard protocols and 96-99% recall outperforms conventional MRI sensitivity (93%).

In addition, the results show F1-score improvement from 0.891 to 0.976 in the ensemble model, which represents a critical balance between minimizing missed diagnoses (high recall), and reducing unnecessary laparoscopies (high precision). This balance is particularly important given the invasive nature of diagnostic laparoscopy and the consequences of delayed treatment.

The advanced pipeline achieved AUC-ROC values of 0.9298-0.9598 compared to 0.8320-0.8855 in the basic approach. This improvement indicates better discrimination between endometriosis and non-endometriosis cases, more reliable performance across different probability thresholds, and greater area under the precision-recall curve, critical for imbalanced datasets.

The advanced pipeline significantly outperforms previous machine learning approaches to endometriosis diagnosis. Accordingly, the 12.4% higher recall than conventional screening protocols represents a potential reduction in diagnostic delays from 7 years to approximately 3 years.

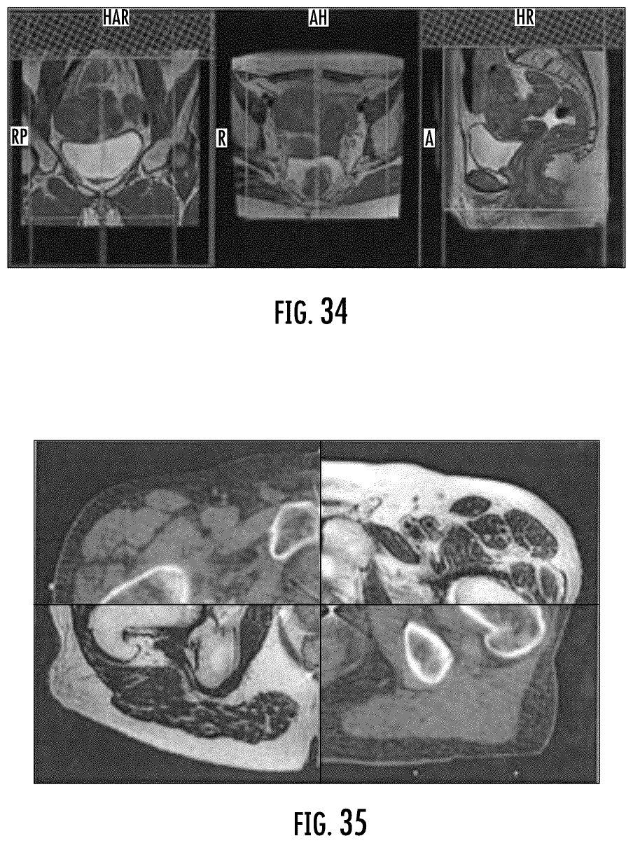

Unlike what is typically done in brain magnetic resonance imaging, pelvic magnetic resonance imaging does not have any sort of skull stripping process applied. Instead, cropping the entire volume of interest of the pelvis is used. FIG. 34 shows an example of a cropped volume of the pelvis.

Image co-registration is the process of applying a series of non-distorting affinity transformations to make an image in the moving space match a template image in the fixed space. This is used to standardize every image that feeds a segmentation model based on a common template volume. It is safe for any clinical condition, since affinity transformations essentially apply zooms and rotations to make the images match, not distorting any lesion.

Here, image co-registration is applied by the system to match new T2 cropped images of the pelvis to a common template image before model inference, in the preprocessing pipeline. FIG. 35 shows a multimodal (CT and MRI) image matching through co-registration.

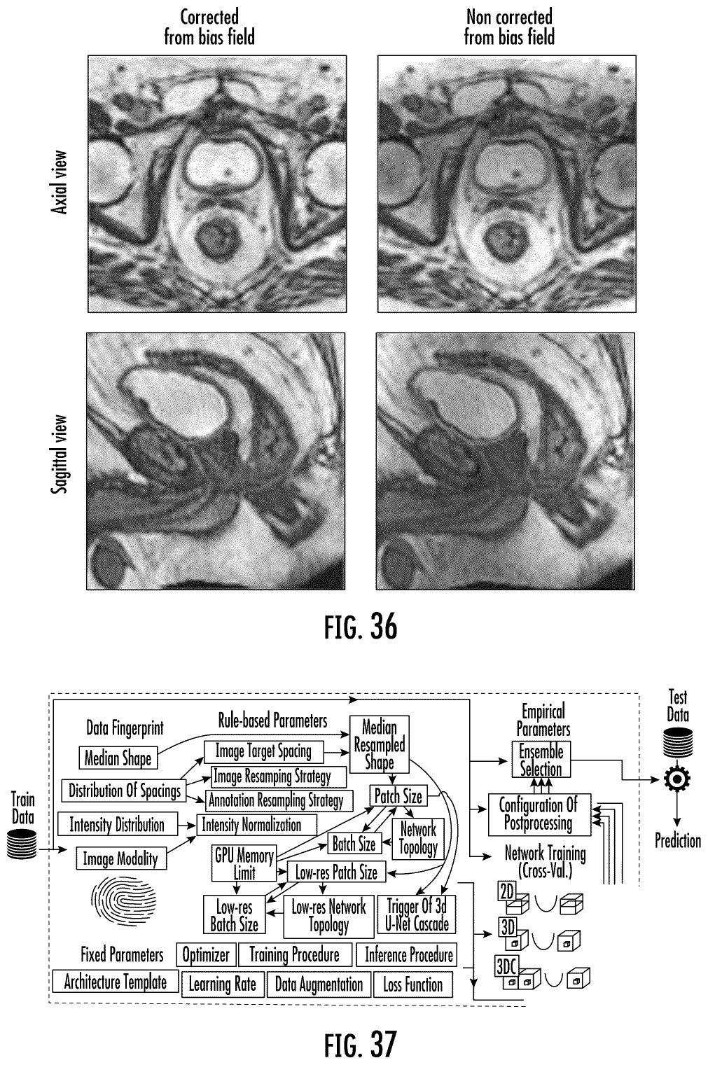

Bias field correction (BFC) is a process of correcting nonuniform intensities generated by the powerful nonuniform magnetic field that generates slices of an MRI volume, causing parts of the image to be brighter or darker than they should be. It is an algorithm applied after the scan, as one of the preprocessing steps, that both: a) estimates the bias filed, and b) corrects for it mathematically. FIG. 36 shows a comparison between a not corrected image, and a corrected image of the pelvis.

Denoising is the process of using a spatially adaptive filter to attenuate noise registered in the image during scan. This filter was described by Manjón et al. (2010), and is built-in to the ANTsPy package.

Image normalization is the process of scaling voxel values of an image to a common range, usually between 0 and 1. This is done by transforming the volume using the formula shown below.

$$Image = \frac{Image - min ( Image )}{max ( Image ) - min ( Image )}$$

This is a required step for properly adjusting model weights without overfitting or any problem related to a non-standardized range of voxel values.

For semantic segmentation, the use of the nnU-net framework Isensee et al. (2021) may be implemented. It is a powerful open source tool specifically designed to provide a robust self-configuring baseline for semantic segmentation in biomedical imaging, primarily leveraging variations of the successful U-Net architecture. Its core principle is automation as upon analyzing the properties of a given dataset, nnU-Net automatically determines and configures the optimal preprocessing steps, network architecture (including choices between 2D/3D, patch sizes, etc.), training schedule, and inference parameters without requiring dataset-specific manual tuning. FIG. 37 shows the architecture overview of nnU-Net and how it works.

Both the images and their corresponding segmentation maps are used throughout the steps of this preprocessing pipeline. Algorithms such as BFC, for example, can be applied either to the entire image or only to the volume of interest (defined by the segmentation map), which significantly reduces the computational cost of processing our entire volume dataset.

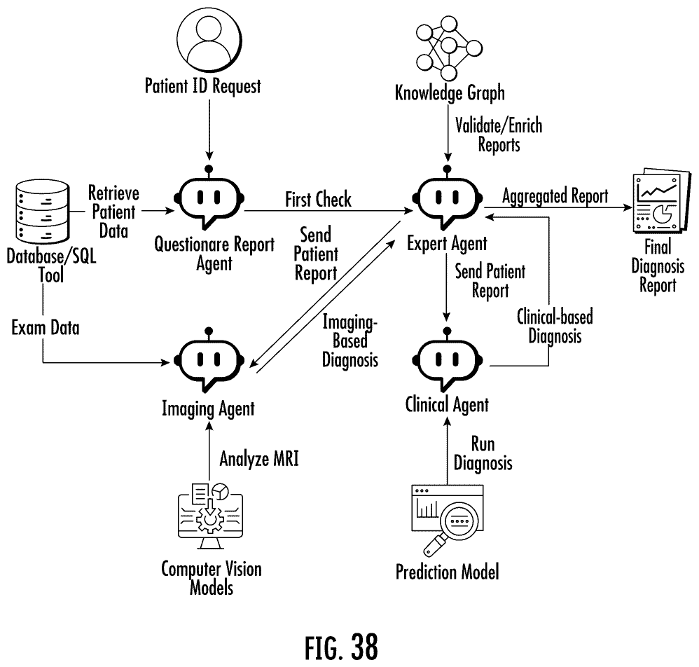

The system may be implemented using a graphical user interface (“GUI”) as depicted in FIG. 38. The GUI includes an Expert Agent that can function independently as an interactive chatbot that addresses user queries regarding endometriosis. It provides accurate, evidence-based responses by leveraging a tailored knowledge graph-including the latest clinical guidelines, research findings, and treatment protocols.

For example, patients can ask specific questions about symptoms, diagnosis, and treatment options, receiving clear, accessible explanations. Clinicians can use the chatbot during consultations to quickly retrieve information on best practices, emerging research, or historical case studies. Advantages of the Expert Agent include immediate access to expert knowledge, reduced information overload through streamlined data presentation, and scalable engagement available any time of day.

The GUI may also have a Report Agent (also referred to as the Questionnaire Report Agent) that can operate as a standalone tool to query and compile a patient's historical clinical data from the database. It is configured to produce a detailed, structured report that enables physicians to review past records, trends, and key data points before appointments. Physicians are able to access the historical report to familiarize themselves with a patient's previous diagnoses, treatments, and outcomes. The report highlights critical trends and anomalies in the patient's data to facilitate informed decision-making during consultations. Advantages include enhanced clinician preparation with comprehensive historical data, consistency through standardized clinical terminologies, and transparent documentation via detailed reasoning traces.

The Questionnaire Report Agent retrieves patient data from the database using an SQL tool. It compiles data that includes demographics, clinical history, lab values, surgical records, and questionnaire responses-into a structured, multi-section report. The responsibilities of this agent include data querying and breaking down complex queries into segments, ensuring capture of all essential data and flagging any missing or inconsistent fields. The Questionnaire Report Agent also assembles retrieved data into a clear report using standardized clinical terminologies (e.g., ICD codes, SNOMED CT), logs the rationale behind data aggregation and detected anomalies, creating an auditable trail, and provides a draft report for clinician review, allowing manual corrections and annotations prior to further processing.

A Clinical Agent of the system processes the refined report from the Questionnaire Report Agent. It employs its predictive model to analyze clinical and questionnaire data, yielding a diagnostic suggestion for endometriosis along with key attention points. The Clinical Agent implements a predictive model to the structured clinical data, outputting a diagnostic suggestion, operates exposing internal metrics (e.g., feature importance, model confidence), focusing on clear and complete clinical insights, and summarizes the analysis in standardized medical language, detailing which clinical trends and data points influenced the decision. In addition, the Clinical Agent is configured to provide an interface for physicians to validate or challenge the output, capturing feedback for ongoing improvements.

The system also includes an Imaging Agent. The Imaging Agent independently processes MRI imaging data and any available surgical descriptions. It uses an advanced imaging model to extract diagnostic features, quantify risk, and provide an imaging-based diagnostic output. The Imaging Agent analyzes MRI data to detect and quantify key imaging markers such as lesions and tissue anomalies, functions independently from the Clinical Agent to ensure unbiased imaging analysis, produces a detailed log of imaging findings using standardized radiological terminologies, and provides radiologists an opportunity to review and flag discrepancies with clinical expectations.

An Endometriosis Expert Agent of the system serves as the quality controller and integrator within the multi-agent system and can also function as an independent assistive chatbot. It validates outputs from the Clinical and Imaging Agents and enriches the final report using its tailored knowledge graph. In particular, the Endometriosis Expert Agent reviews outputs and reasoning traces from both the Clinical and Imaging Agents, checking for inconsistencies or errors. The Endometriosis Expert Agent integrates current clinical guidelines, research findings, and treatment protocols from its knowledge graph to enhance the diagnostic report. The inputs are merged into a cohesive, enriched report that includes risk stratification and actionable recommendations. The Endometriosis Expert Agent includes standalone Chatbot functionality to engage with users (both patients and clinicians) to provide on-demand, expert advice on endometriosis. In addition, it includes human feedback integration and captures final clinician feedback to continuously improve its knowledge base and response accuracy.

In a particular aspect of the operation of the system, the workflow begins with the input of a patient ID, which triggers the diagnostic process. The Questionnaire Report Agent retrieves patient data and generates a structured report complete with a detailed reasoning trace and standardized clinical terminologies. This report can also be used independently as a historical review tool for clinicians before appointments. The initial report is reviewed by the Endometriosis Expert Agent to ensure data consistency and completeness. Any discrepancies or gaps are flagged, and suggestions for refinement are provided.

The refined report is independently sent to the Clinical Agent, which analyzes the clinical and questionnaire data to generate a diagnostic suggestion along with its reasoning trace, and the Imaging Agent, which processes MRI and surgical data to produce an imaging-based diagnostic output and corresponding reasoning trace. Both agents operate in parallel without inter-agent communication, ensuring unbiased independent assessments.

The Endometriosis Expert Agent receives the outputs and reasoning traces from both diagnostic agents. It validates the findings, enriches the information using its knowledge graph, and prepares a comprehensive final report that includes contextual insights, risk stratification, and recommended next steps. The final enriched report is presented to physicians for review. Their feedback is captured and used to refine the models, reasoning processes, and knowledge graph, ensuring continuous system enhancement.

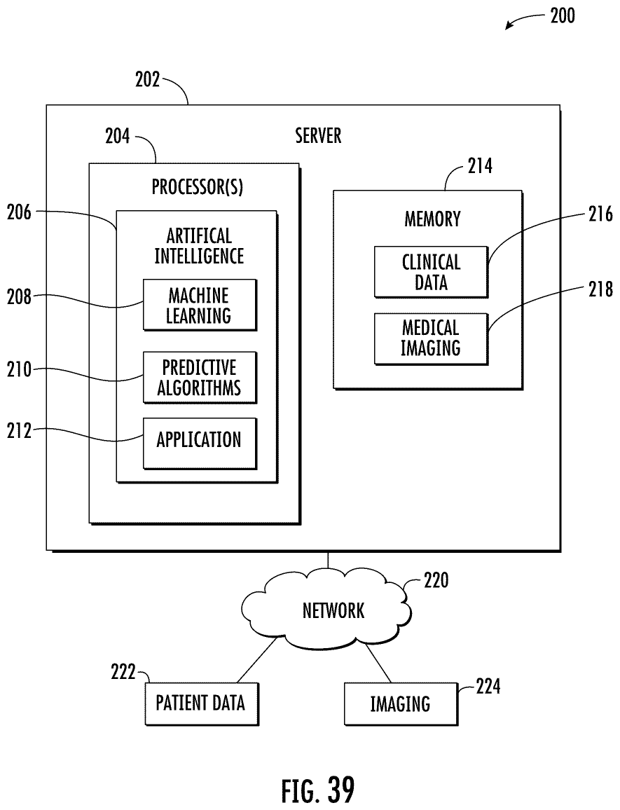

Turning to FIG. 39, a block diagram of the system for endometriosis diagnosis 200 is depicted in accordance with an illustrative embodiment. The system 200 comprises a server 202, at least patient data 222 and patient pelvic medical imaging 224. The server 202 comprises processors 204, and artificial intelligence 206. Artificial intelligence 206 includes machine learning 208, predictive algorithms 210, and an application 212. Machine learning 208 and predictive algorithms 210, in addition to other elements and programs, such as application 212, make the server 202 a special purpose computer for a system for endometriosis diagnosis.

Predictive algorithms 210 may be configured for use by machine intelligence 206 as described above to use patterns and anomalies, as well as numerical values, statistical weights for determining probabilities of a positive endometriosis diagnosis for the patient. The machine or artificial intelligence of the system may also configured to facilitate the exchange of patient data 222 and medical imaging 224 over the network 120. The memory 214 comprises stored clinical data 216 and medical imaging 218 that may be used to further train the system to improve accuracy of endometriosis diagnosis.

In addition, the above system and methods for endometriosis diagnosis may be expanded for evaluating chronic pelvic pain through principles of neuropelveology. Neuropelveology, a subspecialty combining neuroanatomy and pelvic disorders, offers a systematic clinical pathway for diagnosing and differentiating chronic pelvic pain that may arise from nerve dysfunction, entrapment, or irritation, independent of (but potentially coexisting with) conditions like endometriosis.

The objective of the neuropelveology assessment is to systematically and reproducibly identify neuropathic pain components in pelvic pain patients, improve diagnostic specificity, and optimize patient stratification for targeted treatment interventions, including but not limited to minimally invasive surgery, neuromodulation, or conservative therapy.

The neuropelveology assessment involves a structured patient history intake emphasizing neurogenic symptoms (e.g., dermatomal pain, paresthesia, motor deficits). The neuropelveology assessment also includes integration in clinical documentation or stand alone assessment targeted physical examination techniques, including sensory mapping, pelvic nerve palpation, and neurodynamic testing, focused on the sacral plexus, pudendal nerve, obturator nerve, sciatic nerve, and related pelvic nerve structures. The neuropelveology assessment may include optional integration of symptom mapping into a structured diagnostic algorithm that distinguishes somatic, visceral, and neuropathic pain origins. Optional adjuncts (non-mandatory) may include imaging (e.g., MR neurography) or nerve conduction studies for further diagnostic confirmation, but the assessment method itself is clinical and non-invasive.

The clinical utility of the neuropelveology assessment enables early identification of neuropathic components in patients with otherwise unexplained or refractory chronic pelvic pain, and allows differential diagnosis between visceral pain sources (such as endometriosis or adenomyosis) and primary neuropathic pelvic syndromes. The neuropelveology assessment may guide clinical decision-making on further interventions, including targeted pelvic nerve decompression, laparoscopic neurosurgical procedures, or neuromodulation therapy. Moreover, the neuropelveology assessment enhances comprehensive care by addressing neurogenic pain pathways often overlooked in conventional pelvic pathology diagnostics.

The neuropelveology assessment methodology is a complementary module to expand the diagnostic spectrum for chronic pelvic pain. It provides a clinically validated framework that may be integrated into the endometriosis diagnosis platform, contributing to expanded differential diagnosis capabilities.

Many modifications and other embodiments of the invention will come to the mind of one skilled in the art having the benefit of the teachings presented in the foregoing descriptions and the associated drawings. Therefore, it is understood that the invention is not to be limited to the specific embodiments disclosed, and that modifications and embodiments are intended to be included within the scope of the appended claims.

That which is claimed is:

1. A computing system for endometriosis diagnosis, the computing system comprising:
at least one processor; and
a memory storing instructions that, when executed by the at least one processor, causes the system to:
execute a machine learning algorithm to analyze training data comprising pelvic medical images and clinical data that include both patients diagnosed with endometriosis, and patients not diagnosed with endometriosis; and
determine a probability of a hypothetical patient belonging to a group with endometriosis based on a plurality of rules developed from patterns learned from the training data.

2. The computing system of claim 1, further comprising instructions that, when executed by the at least one processor, causes the system to:
acquire clinical data for a particular patient;
acquire pelvic medical imaging for the particular patient; and
determine a probability of an endometriosis diagnosis for the particular patient using the plurality of rules from the machine learning algorithm and the clinical data and pelvic medical imaging for the particular patient.

3. The computing system of claim 2, wherein the clinical data comprises lower back pain, bloating, dysmenorrhea, fatigue, vaginal touch, infertility, pain before period, dyspareunia, pain during period, and regular stomach pain.

4. The computing system of claim 1, wherein the machine learning algorithm comprises one of logistic regression, Random Forest, and XG Boost.

5. The computing system of claim 1, wherein the medical imaging comprises a magnetic resonance imaging (MRI) image or an ultrasound image.

6. The computing system of claim 5, wherein the MRI image is cropped to capture the volume of interest.

7. The computing system of claim 6, wherein bias field correction is applied to the MRI image.

8. The computing system of claim 7, wherein a spatially adaptive filter is applied to the MRI image to attenuate noise registered in the MRI image during scanning.

9. The computing system of claim 9, wherein voxel values of the MRI image are scaled to a controlled range.

10. The computing system of claim 2, further comprising instructions that, when executed by the at least one processor, causes the system to:
analyze the clinical data for the particular patient to generate a clinical based diagnosis; and
process the medical imaging to produce an imaging based diagnosis.

11. A computer-implemented method of endometriosis diagnosis, the computer-implemented method comprising:
applying, by a computer system, one or more machine learning models to analyze training data comprising pelvic medical images and clinical data that include both patients diagnosed with endometriosis, and patients not diagnosed with endometriosis; and
determining a probability of a hypothetical patient belonging to a group with endometriosis based on a plurality of rules developed from patterns learned from the training data.

12. The computer-implemented method of claim 11, further comprising, by the computer system:
acquiring clinical data for a particular patient;
acquiring pelvic medical imaging for the particular patient; and
determining a probability of an endometriosis diagnosis for the particular patient using the plurality of rules from the machine learning algorithm and the clinical data and pelvic medical imaging for the particular patient.

13. The computer-implemented method of claim 12, wherein the clinical data comprises lower back pain, bloating, dysmenorrhea, fatigue, vaginal touch, infertility, pain before period, dyspareunia, pain during period, and regular stomach pain.

14. The computer-implemented method of claim 11, wherein the machine learning algorithm comprises one of logistic regression, Random Forest, and XG Boost.

15. The computer-implemented method of claim 11, wherein the medical imaging comprises a magnetic resonance imaging (MRI) image or an ultrasound image.

16. The computer-implemented method of claim 15, wherein the MRI image is cropped to capture the volume of interest.

17. The computer-implemented method of claim 11, further comprising:
analyzing the clinical data for the particular patient to generate a clinical based diagnosis; and
processing the medical imaging to produce an imaging based diagnosis.

18. A non-transitory computer-readable storage medium including instructions that, when executed by at least one processor of a computing system, cause the computing system to perform a method comprising:
applying one or more machine learning models configured to analyze training data comprising pelvic medical images and clinical data that include both patients diagnosed with endometriosis, and patients not diagnosed with endometriosis; and
determining a probability of a hypothetical patient belonging to a group with endometriosis based on a plurality of rules developed from patterns learned from the training data.

19. The non-transitory computer-readable storage medium of claim 18, further comprising instructions for:
acquiring clinical data for a particular patient;
acquiring pelvic medical imaging for the particular patient;
and determining a probability of an endometriosis diagnosis for the particular patient using the plurality of rules from the machine learning algorithm and the clinical data and pelvic medical imaging for the particular patient.

20. The non-transitory computer-readable storage medium of claim 19, further comprising instructions for:
analyzing the clinical data for the particular patient to generate a clinical based diagnosis; and
processing the medical imaging to produce an imaging based diagnosis.
